SEQUENCE LISTING (102 sequences)

SEQ ID NO: 1 - protein, 408 aa, synthetic construct
EVQLVESGGGLVQPGGSLRLSCAASGRTFSSNAMGWFRQAPGKGREFVAAISWSGGSTYYLDSVKGRFTISRDNAKNTVYLQMNSLRAEDTAVYYCAGSAGLGYVGDPDAMDYWGKGTQVTVSSSAAGGGGSGGGGSAAALKFQCGQKTLRPQFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKEQSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 2 - protein, 408 aa, synthetic construct
DVQLVESGGGLVQPGGSLRLSCAASGRTFSSNAMGWFRQAPGKGREFVAAISWSGGSTYYLDSVKGRFTISRDNAKNTVYLQMNSLRAEDTAVYYCAGSAGLGYVGDPDAMDYWGKGTQVTVSSSAAGGGGSGGGGSAAALKFQCGQKTLRPQFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKEQSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 3 - protein, 408 aa, synthetic construct
DVQLVESGGGLVQPGGSLRLSCAASGRTFSSNAMGWFRQAPGKGREFVAAISWSGGSTYYLDSVKGRFTISRDNAKNTVYLQMNSLRAEDTAVYYCAGSAGLGYVGDPDAMDYWGKGTQVTVSSSAAGGGGSGGGGSAAALKFQCGQKTLRPQFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGHEQSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 4 - protein, 85 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 5 - protein, 20 aa, synthetic construct
METDTLLLWVLLLWVPGSTG

SEQ ID NO: 6 - protein, 13 aa, Lama glama
GEILTTSARDYDY

SEQ ID NO: 7 - protein, 15 aa, Lama glama
SRRQTLYTRTQEYRD

SEQ ID NO: 8 - protein, 15 aa, Lama glama
SRRQTLYTRSQEYND

SEQ ID NO: 9 - protein, 16 aa, Lama glama
RVPSYYFGSYGRSSDY

SEQ ID NO: 10 - protein, 19 aa, Lama glama
DLTIKKTHTIAVVTTRDDY

SEQ ID NO: 11 - protein, 11 aa, Lama glama
KRGLGPWQYEY

SEQ ID NO: 12 - protein, 13 aa, Lama glama
AEKYSSRSERYNY

SEQ ID NO: 13 - protein, 13 aa, Lama glama
AESYSSRSDRYKY

SEQ ID NO: 14 - protein, 13 aa, Lama glama
AESYSSRSERYNY

SEQ ID NO: 15 - protein, 13 aa, Lama glama
AESYSSRSERYNY

SEQ ID NO: 16 - protein, 13 aa, Lama glama
AESYSTRSERYNY

SEQ ID NO: 17 - protein, 13 aa, Lama glama
ADSYSCRSETYNY

SEQ ID NO: 18 - protein, 17 aa, Lama glama
SYGLVATMEMEMSDFSS

SEQ ID NO: 19 - protein, 15 aa, Lama glama
QGGIATVADPDAYDY

SEQ ID NO: 20 - protein, 15 aa, Lama glama
QGGMATVADPDAYDY

SEQ ID NO: 21 - protein, 15 aa, Lama glama
QGGMATVSDTEAYDD

SEQ ID NO: 22 - protein, 15 aa, Lama glama
QGGIATMADMDAYDY

SEQ ID NO: 23 - protein, 15 aa, Lama glama
SAGLGYVGDPDAMDY

SEQ ID NO: 24 - protein, 15 aa, Lama glama
AYTWNTRTPDGLVDF

SEQ ID NO: 25 - protein, 15 aa, Lama glama
AYTIAVVTAMREYDF

SEQ ID NO: 26 - protein, 10 aa, Lama glama
DPRDSGFYDY

SEQ ID NO: 27 - protein, 16 aa, Lama glama
RHDSRGTYYSSRGYDY

SEQ ID NO: 28 - protein, 18 aa, Lama glama
RSSAFSSGIYYREGSYAY

SEQ ID NO: 29 - protein, 7 aa, Lama glama
GRTASSY

SEQ ID NO: 30 - protein, 7 aa, Lama glama
GRTSDNY

SEQ ID NO: 31 - protein, 7 aa, Lama glama
GRTSDNY

SEQ ID NO: 32 - protein, 7 aa, Lama glama
GGVFTPN

SEQ ID NO: 33 - protein, 7 aa, Lama glama
GSIFSIN

SEQ ID NO: 34 - protein, 7 aa, Lama glama
GYRFGIN

SEQ ID NO: 35 - protein, 7 aa, Lama glama
GRSISNY

SEQ ID NO: 36 - protein, 7 aa, Lama glama
GRSFSSY

SEQ ID NO: 37 - protein, 7 aa, Lama glama
GRSFSRH

SEQ ID NO: 38 - protein, 7 aa, Lama glama
GRSFSRY

SEQ ID NO: 39 - protein, 7 aa, Lama glama
GRRGSSY

SEQ ID NO: 40 - protein, 7 aa, Lama glama
GLTFSRH

SEQ ID NO: 41 - protein, 7 aa, Lama glama
GRTFDSY

SEQ ID NO: 42 - protein, 7 aa, Lama glama
GRTFSSV

SEQ ID NO: 43 - protein, 7 aa, Lama glama
GRTFSSV

SEQ ID NO: 44 - protein, 7 aa, Lama glama
GRTFSNY

SEQ ID NO: 45 - protein, 7 aa, Lama glama
GRTFSSV

SEQ ID NO: 46 - protein, 7 aa, Lama glama
GRTFSSN

SEQ ID NO: 47 - protein, 7 aa, Lama glama
GLTFSNY

SEQ ID NO: 48 - protein, 7 aa, Lama glama
GQTLSNY

SEQ ID NO: 49 - protein, 7 aa, Lama glama
GRTFSNY

SEQ ID NO: 50 - protein, 7 aa, Lama glama
GRTFSTY

SEQ ID NO: 51 - protein, 7 aa, Lama glama
GRTFSSL

SEQ ID NO: 52 - protein, 5 aa, Lama glama
GTAGS

SEQ ID NO: 53 - protein, 6 aa, Lama glama
SWSGTS

SEQ ID NO: 54 - protein, 6 aa, Lama glama
SWSGTS

SEQ ID NO: 55 - protein, 5 aa, Lama glama
TSDGT

SEQ ID NO: 56 - protein, 5 aa, Lama glama
TSGGR

SEQ ID NO: 57 - protein, 5 aa, Lama glama
TSGGS

SEQ ID NO: 58 - protein, 6 aa, Lama glama
TSSGLS

SEQ ID NO: 59 - protein, 6 aa, Lama glama
TRSGLD

SEQ ID NO: 60 - protein, 6 aa, Lama glama
TWSGLD

SEQ ID NO: 61 - protein, 6 aa, Lama glama
TWSGLD

SEQ ID NO: 62 - protein, 6 aa, Lama glama
TWSGLD

SEQ ID NO: 63 - protein, 6 aa, Lama glama
TCNGLN

SEQ ID NO: 64 - protein, 6 aa, Lama glama
SWSGGS

SEQ ID NO: 65 - protein, 6 aa, Lama glama
NWSGGS

SEQ ID NO: 66 - protein, 6 aa, Lama glama
SWSGGS

SEQ ID NO: 67 - protein, 6 aa, Lama glama
SWSGGN

SEQ ID NO: 68 - protein, 6 aa, Lama glama
NWSGGS

SEQ ID NO: 69 - protein, 6 aa, Lama glama
SWSGGS

SEQ ID NO: 70 - protein, 6 aa, Lama glama
SKSGGN

SEQ ID NO: 71 - protein, 6 aa, Lama glama
SRVGGS

SEQ ID NO: 72 - protein, 6 aa, Lama glama
MWSAAS

SEQ ID NO: 73 - protein, 6 aa, Lama glama
YWRDGS

SEQ ID NO: 74 - protein, 5 aa, Lama glama
GSDSS

SEQ ID NO: 75 - protein, 124 aa, Lama glama
EVQLVESGGGLVQAGGSLRLSCAASGRTFSSNAMGWFRQAPGKEREFVAAISWSGGSTYYLDSVKGRFTISRDNAKNTVYLQMNSLKPEDTAVYYCAGSAGLGYVGDPDAMDYWGKGTQVTVSS

SEQ ID NO: 76 - protein, 124 aa, Lama glama
EVQLVESGGGLVQPGGSLRLSCAASGRTFSSNAMGWFRQAPGKGREFVAAISWSGGSTYYLDSVKGRFTISRDNAKNTVYLQMNSLRAEDTAVYYCAGSAGLGYVGDPDAMDYWGKGTQVTVSS

SEQ ID NO: 77 - protein, 124 aa, Lama glama
EVQLVESGGGLVQAGGSLRLSCAASGQTLSNYVMGWFRQAPGKEREFVAVISRVGGSTSYADSAKGRFTISRDNAKNTVYLQMNSLKPEDTAVYYCAAAYTIAVVTAMREYDFWGQGTQVTVSS

SEQ ID NO: 78 - protein, 126 aa, Lama glama
AVQLVESGGRLVKAGASLRLSCAASGRTFSSLPMAWFRQAPGKEREFVAFIGSDSSTLYTSSVRGRFTISRDNGKNTVYLQMMNLKPEDTAVYYCAARSSAFSSGIYYREGSYAYWGQGTQVTVSS

SEQ ID NO: 79 - protein, 125 aa, Lama glama
QVQLVESGGGLVQAGGSLRLSCTASGRTFSTYALGWFRQVPGKGREFIAVIYWRDGSSLYSDSVKGRFTISKDNAKNTVYLQMNSLKPEDTAVYYCANRHDSRGTYYSSRGYDYWGQGTQVTVSS

SEQ ID NO: 80 - protein, 122 aa, Lama glama
EVQLVESGGGLVQAGESLRLSCAISGRTASSYVVAWFRQAPGEVGRQFVTRIGTAGSTRYADSVKGRFVISRDDAKNTVYLQMNSLKPEDTAFYYCAIGEILTTSARDYDYWGQGTQVTVSS

SEQ ID NO: 81 - protein, 124 aa, Lama glama
EVQLVESGGGLVQAGGSLRLSCATSGRTSDNYNMGWFRQTPGKAREFVSAISWSGTSTYYADSVRGRFTVSRDNAKNTAYLQMDSLKPEDTAVYYCAASRRQTLYTRTQEYRDWGQGTQVTVSS

SEQ ID NO: 82 - protein, 124 aa, Lama glama
EVQLVESGGGLVQAGGSLRLSCATSGRTSDNYNMGWFRQAPGKAREFVSASSWSGTSTYYADSVRGRFTVSRDNAKNTVYLQMDSLKPEDTAVYYCAASRRQTLYTRSQEYNDWGQGTQVTVSS

SEQ ID NO: 83 - protein, 124 aa, Lama glama
EVQLVESGGGLGQPGGSLNLSCVASGGVFTPNTMGWYRQAPGKQRELVATATSDGTTNYADSVKGRFTISRDNAKKTVYLQMTSLKPEDTAVYYCNIRVPSYYFGSYGRSSDYWGQGTQVTVSS

SEQ ID NO: 84 - protein, 124 aa, Lama glama
EVQLVESGGGLVQTGSSLRLSCAASGLTFSNYVVGWFRQAPGKEREFVAIISKSGGNTYYAESVKGRFTASRENAKNTAYLQMNSLQPEDTAVYYCAAAYTWNTRTPDGLVDFWGQGTQVTVSS

SEQ ID NO: 85 - protein, 119 aa, Lama glama
EVQLVESGGGLVQAGGSLRLSCAASGRTFSNYRMAWFRQAPGKGREFVARIMWSAASTTYADSVKGRFTISRDNAKNTVYLQMNSLKPEDTAVYYCAADPRDSGFYDYWGQGTQVTVSS

SEQ ID NO: 86 - protein, 124 aa, Lama glama
EVQLVESGGGLVQAGGSLRLSCAASGQTLSNYVMGWFRQAPGKEREFVALISRVGGSTSYADSVKGRFTISRDNAKNTVYLQMNSLKPEDTAVYYCAAAYTIAVVTAMREYDFWGQGTQVTVSS

SEQ ID NO: 87 - protein, 122 aa, Lama glama
EVQLVESGGGLVQAGGSLRLSCAASGRSFSSYVMGWFRQAPGKERMFVAAITRSGLDTYNSDSMKGRFTISRDNAKNTVYLQMNSLKPEDTAVYYCAAAESYSSRSDRYKYWGQGTQVTVSS

SEQ ID NO: 88 - protein, 122 aa, Lama glama
EVQLVESGGGLVQAGGSLRLSCAGSGRSISNYVMGWFRQAPGKERNYVAAITSSGLSTYNSDSTKDRFTISRDNAKNTVYLQMNSLKPEDTAVYYCAAAEKYSSRSERYNYWGQGTQVTVSS

SEQ ID NO: 89 - protein, 122 aa, Lama glama
EVQLVESGGGLVQAGGSLRLSCAASGRSFSRHVMGWFRQAPGKERAFVAAITWSGLDTYSSDSLKGRFTISRDNAKNTVYLQMNSLKPEDTAVYYCAAAESYSSRSERYNYWGQGTQVTVSS

SEQ ID NO: 90 - protein, 122 aa, Lama glama
EVQLVESGGGLVQAGGSLRLSCAASGLTFSRHVMGWFRQAPGKERAFVATITCNGLNTYSSYSLKGLFTISKDNANNAVYLLMNRLKPDDTVVYYCAAADSYSCRSETYNYCGPGTQVTVSS

SEQ ID NO: 91 - protein, 122 aa, Lama glama
EVQLVESGGGLVQAGGSLRLSCAASGRSFSRHVIGWFRQAPGKERAFVAAITWSGLDTYSSDSLKGRFTISRDNAKNTVYLQMNSLKPEDTAVYYCAAAESYSSRSERYNYWGQGTQVTVSS

SEQ ID NO: 92 - protein, 122 aa, Lama glama
EVQLVESGGGLVQAGGSLRLSCAASGRSFSRYVMGWFRQGPGKERNFVAAITWSGLDTYSSDSLKGRFTISRDNAKNTVYLQMNSLKPEDTAVYYCAAAESYSSRSERYNYWGQGTQVTVSS

SEQ ID NO: 93 - protein, 122 aa, Lama glama
EVQLVESGGGLVQAGGSLRLSCAASGRRGSSYVMGWFRQGSGNSRDFVAAITWSGLDTYNSDSLKGRFTISRDNAKNTVYLQMNSLKPEDTAVYYCAAAESYSTRSERYNYWGQGTQVTVSS

SEQ ID NO: 94 - protein, 127 aa, Lama glama
EVQLVESGGGLVQAGGSLRLSCAASGSIFSINAMGWYRQAPGKQRDLVADITSGGRTNYRDSVKGRFTISRDNAKNAVYLQMNSLKPEDTAVYYCTADLTIKKTHTIAVVTTRDDYWGQGTQVTVSS

SEQ ID NO: 95 - protein, 126 aa, Lama glama
EVQLVESGGGLVQAGGSLTLSCAASGRTFDSYAMGWFRQAPGKEREFVAAISWSGGSTYYADSVKGRFTISRDNAKNTVYLQMNTLKPEDTAVYYCAASYGLVATMEMEMSDFSSWGQGTQVTVSS

SEQ ID NO: 96 - protein, 124 aa, Lama glama
EVQLVESGGGLVQAGGSLRLSCAASGRTFSSVAMGWFRQAPGKEREFVAAINWSGGSTYYLDSVKGRFTISRDNAKNTVYLQMNSLKPEDTAVYYCAGQGGIATVADPDAYDYWGQGTQVTVSS

SEQ ID NO: 97 - protein, 119 aa, Lama glama
EVQLVESGGGLVQAGGSLRLSCAASGYRFGINAMGWYRQAPGKRRELVAIITSGGSTNYTDSVKGRFSISRDNAKNTVYLQMNSLKPEDTAVYYCNQKRGLGPWQYEYWGQGTQVTVSS

SEQ ID NO: 98 - protein, 124 aa, Lama glama
EVQLVESGGGLVQAGGSLRLSCAASGRTFSSVAMGWFRQAPGKEREFVAAISWSGGSTYYLDSVKGRFTISRDNAKNTVSLQMNSLKPEDTAVYYCAGQGGMATVADPDAYDYWGQGTQVTVSS

SEQ ID NO: 99 - protein, 124 aa, Lama glama
EVQLVESGGGLMQAGASLRLSCVASGRTFSSVAMGWFRQAPGKEREFVAAINWSGGSTYYPDSVKGRFTISRDNAQNTVYLQMNSLKPEDTAVYYCAGQGGIATMADMDAYDYWGQGTQVTVSS

SEQ ID NO: 100 - protein, 124 aa, Lama glama
EVQLVESGGGLVQAGGSLRLSCAASGRTFSNYAMGWFRQAPGKEREFVAAISWSGGNTYYADSVKGRFTISRDNAKNTLYLQMNSLKPEDTAVYYCAAQGGMATVSDTEAYDDWGQGTQVTVSS

SEQ ID NO: 101 - protein, 126 aa, Lama glama; identical to SEQ ID NO: 95

SEQ ID NO: 102 - protein, 462 aa, synthetic construct
METDTLLLWVLLLWVPGSTGDGSSAWSHPQFEKGSSAWSHPQFEKEFENLYFQSEVQLVESGGGLVQAGGSLRLSCAASGRTFSSNAMGWFRQAPGKEREFVAAISWSGGSTYYLDSVKGRFTISRDNAKNTVYLQMNSLKPEDTAVYYCAGSAGLGYVGDPDAMDYWGKGTQVTVSSSAAGGGGSGGGGSAAALKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

(19) United States
(12) Patent Application Publication
(10) Pub. No.: US 2026/0125665 A1
(43) Pub. Date: May 7, 2026

(54) TARGETING AND LYSING MICROTHROMBI ASSOCIATED WITH ABERRANT BLOOD CLOTTING

(71) Applicant: TargED Biopharmaceuticals B.V., Utrecht (NL)

(72) Inventors: Steven DE MAAT, Bunschoten-Spakenburg (NL); Coen MAAS, Utrecht (NL)

(21) Appl. No.: 19/371,058

(22) Filed: Oct. 28, 2025

Related U.S. Application Data
(63) Continuation of application No. PCT/EP2024/087672, filed on Dec. 19, 2024.

(30) Foreign Application Priority Data
Dec. 19, 2023 (EP) 23218291.5

(51) Int. Cl. C12N 9/72; A61K 31/195; A61K 38/00; A61K 38/55; A61P 7/02; C07K 16/36; C07K 16/40

(52) U.S. Cl. CPC C12N 9/6462; A61K 31/195; A61K 38/55; A61P 7/02; C07K 16/36; C07K 16/40; C12Y 304/21031; A61K 38/00; C07K 2317/569; C07K 2319/00

(57) ABSTRACT

The present invention provides means and methods for treating MVT and associated and/or similar conditions. The invention therefore provides hybrid proteins for targeted delivery of plasminogen activators to platelet-VWF complexes, or alternatively to the site where these are located, in a fibrin-independent manner. The invention further provides therapeutic methods for conditions that can be prevented or treated by local delivery/stimulation of plasminogen activation to sites of microvascular occlusion.

### CROSS-REFERENCE TO ELECTRONIC SEQUENCE LISTING

The application contains a Sequence Listing which has been submitted electronically in .XML format and is hereby incorporated by reference in its entirety. Said .XML copy, created on Dec. 17, 2024, is named “AMS-202305EPPC Sequence listing.xml” and is 93,282 bytes in size. The sequence listing contained in this. XML file is part of the specification and is hereby incorporated by reference herein in its entirety.

### FIELD OF THE INVENTION

The present invention relates to the field of diseases or conditions associated with undesired blood clotting, in particular microvascular thrombosis, such as thrombotic thrombocytopenia purpura. In particular, the invention relates to a hybrid protein comprising a (urokinase) catalytic domain linked to an antibody-like molecule, in particular a VHH, capable of facilitating the lysis of microthrombi. In a particular embodiment the VHH binds to von Willebrand factor (VWF), enabling the linked urokinase catalytic domain to drive the conversion of plasminogen to plasmin, which then degrades fibrin and/or VWF into smaller fragments. The invention thus provides means and methods for treating microvascular thrombosis and associated conditions.

### BACKGROUND ART

Primary hemostasis is a procoagulation clot forming process associated with the initiation and formation of the platelet plug, in which the binding of platelets to vWF is of importance. Secondary hemostasis is also a procoagulation clot forming process and it is associated with the formation of fibrin via the intrinsic and extrinsic coagulation cascades. In healthy people, homeostatic balance exists between procoagulant forces and anticoagulant and fibrinolytic forces. Numerous genetic, acquired, and environmental factors can tip the balance in favor of coagulation, leading to the pathologic formation of thrombi in veins (eg, deep venous thrombosis [DVT]), arteries (eg, myocardial infarction, ischemic stroke), or cardiac chambers. Thrombi can obstruct blood flow at the site of formation or detach and embolize to block a distant blood vessel (eg, pulmonary embolism, embolic stroke). In here we will focus on microvascular thrombosis.

Microvascular thrombosis (MVT) is characterized by the formation of microvascular platelet aggregates. They are minimally composed of platelets and VWF. This becomes clear in thrombotic thrombocytopeniaurpura (TTP), where platelet- and VWF-rich, but fibrin-poor microthrombi obstruct microvasculature with life-threatening consequences. Thus, fibrin, which is seen in macrovascular thrombosis, is not per se required for these microvascular obstructions. MVT is a shared feature between several disease states including thrombotic thrombocytopenia purpura, hemolytic uremic syndrome, antiphospholipid antibody syndrome and complement-mediated thrombotic microangiopathy (George et al., 2014, N Engl J Med. 371 (7): 654-66). In severe cases of MVT, multi-organ failure can occur with lethal consequences. Even in less severe cases, organ damage may occur, reducing both the quality of life, as well as the life expectancy of the patient. Recent studies suggest that microvascular disease underlies cardiovascular disease/events in patients in the more generalized population with cardiovascular disease that do not show overt signs of macrovascular obstruction on radiological examination. This is ultimately thought to cause heart failure, in particular in females.

Patients with TTP experience attacks of microvascular thrombosis, when platelets form complexes with ultra-large multimers of von Willebrand Factor (VWF). This is the result of severely decreased activity of the enzyme ADAMTS13 (a disintegrin and metalloproteinase with a thrombospondin type I motif, member 13). ADAMTS13 normally regulates the thrombogenicity of VWF by enzymatically reducing its multimer size. Hereto, VWF needs to unfold from its globular form into an unrolled conformation, thereby exposing its A2 domain for proteolysis. The majority of TTP patients suffer from neutralizing autoantibodies against AD AMTS 13. For a small subgroup, mutations in ADAMTS13 have been described that lead to deficiency (Upshaw-Shulman syndrome).

Current TTP therapy involves extensive plasma exchange to deplete inhibitory antibodies and restore ADAMTS13 activity at the same time. However, persistent autoantibodies impede elimination of microthrombi. This makes therapy time-consuming and very costly (Fijnheer et al., Ned Tijdsch Hematol 2016; 13 (1): 18-24).

Besides AD AMTS 13, VWF can be cleaved by the enzyme plasmin (Berkowitz et al., J Clin Invest 1987 February; 79 (2): 524-31). We previously identified that systemic plasminogen activation (with streptokinase) was therapeutic in a mouse model for TTP, suggesting that plasmin can act as a functional alternative to ADAMTS13 (Tersteeg et al., 2014, Circulation, 129 (12): 1320-31). Although plasmin (ogen) can directly bind to unrolled (unfolded) VWF, natural plasminogen activators—such as tissue plasminogen activator (tPA) and urokinase plasminogen activator (uPA)—cannot. The natural targets of tPA and uPA are fibrin and the endothelial cell receptor uPAR, respectively. Furthermore, microthrombi in TTP are fibrin-poor and it is uncertain whether fibrin is a prerequisite for other types of MVT. This renders these molecules (i.e. tPA, uPA) that are generally used as thrombolytic agents in the treatment of macrovascular disease ineffective in treating MVT. Summary of the invention

The invention provides hybrid proteins for targeted delivery of plasminogen activators to platelet-VWF complexes, or alternatively to the site where these are located, in a fibrin-independent manner. The invention further provides the use of the hybrid proteins for the treatment of MVT and associated and/or similar conditions, and methods for conditions that can be prevented or treated by local delivery/stimulation of plasminogen activation to sites of microvascular occlusion.

### FIGURES

In FIG. 1 results are shown indicating susceptibility of Micro lyse variants to cleavage by thrombin. Microlyse variant 1.1 contains a glutamine at position 153, while Microlyse variant 1.0 contains an arginine at position 153.

FIG. 2 shows the EpiMatrix Protein Immunogenicity Scale of ML 1.0 and ML 1.1.

FIG. 3a is deconvoluted MS spectra of ML 1.0 intact (before treatment of PNGaseF) in Example 4.

FIG. 3b is deconvoluted MS spectra of ML 1.0 after treatment of PNGaseF in Example 4.

FIG. 4a is a reducing RP-HPLC profile of ML 1.1 in Example 4, with MS identification of each peak described in the legend. Elution time (min) on the X-axis and UV 214 nm signal in Y axis.

FIG. 4b is deconvoluted MS spectra of peaks 1, 5 and 7 from FIG. 4a with peak masses (Da).

FIG. 5 shows the effect of Alteplase infusion on bleeding time in the tail-clip assay in Example 6. The tail-clip assay was performed at 20 minutes (blue), mid-way through the treatment infusion, or at 40 minutes (red), the end of the treatment infusion. The dotted line at 600 seconds indicates the maximum bleeding time allowed before the cessation of the experiment. ****p (0.0001 or not significant (ns) versus the vehicle (0 mg/kg) group using a one-way analysis of variance with Dunnet's multiple comparisons test.

FIG. 6 shows the effect of TGD001 and Alteplase infusion on bleeding time in the tail-clip assay in Example 6. TGD001 and Alteplase (both 2.0 mg/kg) were given as a 10% bolus followed by a 40-minute infusion (remaining 90%). The tail-clip assay was performed at 20 (during the infusion) and 40 minutes (end of infusion). The dotted line at 600 seconds indicates the maximum bleeding time allowed before the cessation of the experiment. ****P (0.0001 or not significant (ns) versus the vehicle (0 mg/kg) group using a one-way analysis of variance with Dunnet's multiple comparisons test.

FIG. 7 shows the effect of bolus TGD001 administration on bleeding time in the tail-clip assay in Example 6. TGD001 was given as a bolus. The tail-clip assay was performed directly after the bolus injection. The dotted line at 600 seconds indicates the maximum bleeding time allowed before the cessation of the experiment. ****P (0.0001 or not significant (ns) versus the vehicle (0 mg/kg) group using a one-way analysis of variance with Dunnet's multiple comparisons test.

FIG. 8 shows the effect of zymogen or activated TGD001 on bleeding time in the tail-clip model of Example 6. Mice received a bolus of TGD001. The dotted line at 600 seconds indicates the maximum bleeding time allowed before the cessation of the experiment. ****p (0.0001 or not significant (ns) versus the vehicle (0 mg/kg) group using a one-way analysis of variance with Dunnet's multiple comparisons test.

FIG. 9 shows the effect of TXA on TGD001's pharmacological activity in the tail-clip model in Example 6. TGD001 (2 mg/kg) was given as a bolus with or without TXA infusion. The tailclip assay was performed directly after the bolus injection. The dotted line at 600 seconds indicates the maximum bleeding time allowed before the cessation of the experiment. ****p (0.0001 or not significant (ns) versus the vehicle (0 mg/kg) group using a one-way analysis of variance with Dunnet's multiple comparisons test.

### DETAILED DESCRIPTION

Thrombotic microangiopathies are a collection of disorders in which catastrophic disseminated thrombosis of the microvasculature is a central feature. In thrombotic thrombocytopenia purpura (TTP), this is caused by functional ADAMTS13 deficiency (either absence of protein or absence of functional protein or both). This is most often the result of autoantibodies against AD AMTS 13, which clear and neutralize the protease (acquired TTP [aTTP]). TTP has an unpredictable episodic nature, and acute attacks are hallmarked by deep consumptive thrombocytopenia: microthrombi that occlude the vasculature, deplete the systemic circulation from platelets. This leads to ischemic injury in various tissues, accompanied by a severe risk of bleeding. The current standard of care for acute aTTP attacks includes plasma exchange to remove autoantibodies and thereby restore ADAMTS13 activity. Long-term therapeutic strategies include immune modulation to reduce autoantibody levels. The interaction between platelet glycoprotein Iba (GPIba) and von Willebrand Factor (VWF) drives the formation of microthrombi in TTP. This does not require platelet activation, coagulation system activation, or fibrin formation. Recently, a bivalent single-chain antibody (VHH; nanobody) that blocks the binding of platelets to the VWF A1 domain for treatment of aTTP was developed. Administration of this nanobody (INN: caplacizumab) in combination with plasma exchange shortens acute TTP attack duration from 5 days to 3 to 4 days. This was demonstrated by a faster normalization of the platelet count and reduced need for plasma exchange (by 39%), which has a large impact on the organ damage that patients sustain during attacks. In essence, shortening the attack duration is very important to the management of TTP.

We previously found evidence for activation of endogenous plasminogen in patients during TTP attacks. We proposed an auxiliary role of plasmin as a VWF-cleaving enzyme in this setting and subsequently demonstrated that systemic plasminogen activation (with a bacterial plasminogen activator) attenuated thrombocytopenia in a murine TTP model. Subsequently, we demonstrated that endogenous plasminogen activation regulates disease severity in a mouse model for aTTP. These findings led us to believe that exploiting the VWF-cleaving properties of plasmin holds therapeutic value. While plasminogen can directly bind to VWF in a lysine dependent manner, neither tissue plasminogen activator (tPA) nor urokinase plasminogen activator (uPA) can bind VWF directly. According to the invention, strategies to bring plasminogen activators to VWF have value for the treatment of TTP.

We have developed a new thrombolytic agent for the destruction of platelet-VWF complexes. In particular, what has been developed is a VWF-targeting plasminogen activator for plasmin-mediated destruction of platelet-VWF complexes that is not critically dependent on the presence of fibrin-, thrombin-, or platelet activation.

According to the invention it has been found that the plasminogen activator, more specifically the urokinase catalytic domain of the plasminogen activator, has at least two elements that may negatively impact the stability and thereby the therapeutic performance. First of all, there is a possible cleavage site of thrombin, resulting in inactivation of the urokinase catalytic domain. This cleavage site has been found to be located at the arginine at position 153 of the urokinase catalytic domain. The cleavage site can be removed by changing the arginine at position 153. For this, an amino acid should be chosen that is not susceptible for cleavage by thrombin. Preferably, the amino acid is structurally and/or chemically similar to arginine. The most similar amino acid is histidine, but histidine has a benzene ring, making it a less suitable candidate because of its impact on the structure. Because of this, other amino acids were considered, glutamine is the most suitable candidate according to the invention. A second effect that was observed to possibly negatively impact the stability of the urokinase catalytic domain is incorrect or incomplete glycosylation, which can seriously impact the half-life time of the protein and may result in immunogenicity problems. The urokinase catalytic domain has been found to comprise an endogenous N-glycosylation site located at the asparagine at position 299. The possibly unwanted endogenous N-glycosylation site may be removed by changing the asparagine at position 299.

Thus the invention provides a polypeptide comprising or consisting of a urokinase catalytic domain, wherein the arginine residue at position 153 is substituted with a different amino acid residue, preferably a glutamine residue. In a further embodiment the polypeptide comprises a substitution of the asparagine residue at position 299. Most preferably the asparagine residue at position 299 is substituted with a glutamine residue. These mutants retain their structure and activity at least to a useful extent. Furthermore, the immunogenicity of these mutants is acceptable for therapeutic use according to our tests (in silico and others). In another embodiment, a polypeptide according to the invention comprise a substitution of the lysine residue at position 297. Preferably the lysine residue at position 297 is substituted with histidine. The term “polypeptide” refers to molecules consisting of a chain of amino acids, wherein the amino acids are linked by means of a covalent (peptide) bond. The term “urokinase catalytic domain” refers to a specific region or segment of the urokinase-type plasminogen activator (uPA) protein. The terms “urokinase-type plasminogen activator” and “urokinase” are used interchangeably and refer to an enzyme that plays a role in the breakdown of blood clots, a process known as fibrinolysis. In particular, the urokinase protein can facilitate the lysis of blood clots (i.e. microthrombi) by promoting the conversion of plasminogen to plasmin, which then degrades fibrin, the protein meshwork that forms blood clots, into smaller fragments. The urokinase protein is composed of several functional domains, each with its unique role in its enzymatic activity. The “catalytic domain” is the part of the urokinase protein responsible for its enzymatic function, specifically the ability to activate plasminogen into plasmin. In particular, urokinase is a serine protease that has the ability to catalyze the activation of plasmin via proteolytic cleavage of plasminogen. The term “residue” refers to a specific individual building block (i.e. an amino acid) within the polypeptide chain. Each amino acid in the chain is referred to as a residue. The term “position” used herewith refers to a specific position of a residue in the amino acid chain of a polypeptide. The number of the residue is assigned starting from the first amino acid at the N-terminus of the polypeptide. The first residue at the N-terminus is assigned number 1, the second residue is assigned number 2, et cetera. The position of the residue as referred to herewith can be for instance counted from the first amino acid in one of the following sequences: SEQ ID No. 1-3, preferably in SEQ ID No. 1. For example, position 153 refers to the 153th amino acid in the polypeptide chain, counted from the N-terminus, preferably in one of the following sequences: SEQ ID No. 1-3, more preferably SEQ ID No. 1. The term “substitution” refers to a specific type of mutation that occurs when an amino acid residue in a polypeptide chain is replaced by a different amino acid residue. For instance, when an arginine residue is replaced with a glutamine residue.

The invention further provides a hybrid protein comprising the polypeptide comprising or consisting of a urokinase catalytic domain, wherein the polypeptide is linked to a specific binding molecule, such as an antibody-like molecule, preferably a VHH. The term “hybrid protein” is defined as a complex of two or more polypeptide sequences or fragments thereof which would not normally be associated, but are coupled together either by fusing the genes (optionally with a peptide linker) which encode them or by chemically cross-linking the component parts. The terms “VHH” and “VHH antibody” are used interchangeably and refer to a single-domain antibody or nanobody, which is a type of antibody derived from camelids (camels, llamas, and alpacas) or cartilaginous fish (such as sharks and rays). Unlike conventional antibodies (IgG antibodies) found in humans and other mammals, which have two heavy chains and two light chains, VHH antibodies have a single polypeptide chain containing a single variable domain derived from the heavy chain. A VHH antibody has a framework region (FR) and a complementarity-determining region (CDR). The “framework region”, is typically comprised of framework regions 1-4 (FR1-FR4), and forms the scaffold of the VHH antibody's variable domain, providing structural support and maintaining the overall shape of the binding site. It comprises relatively conserved amino acid sequences that stabilize the antibody structure and contribute to its folding. The “complementarity-determining regions (CDRs)”, also known in the art as hypervariable regions, are short stretches of amino acids within the variable domain of an antibody. CDRs are highly diverse and responsible for antigen recognition and binding specificity. There are typically three CDRs in the variable domain of VHH antibodies, generally named CDR1, CDR2, and CDR3. The complementarity determining region 3 (CDR3) preferably comprises any of the amino acid sequences listed in SEQ ID NO: 6-28, the complementarity determining region 1 (CDR1) preferably comprises any of the amino acid sequences listed in SEQ ID NO: 29-51, and the complementarity determining region 2 (CDR2) preferably comprises any of the amino acid sequences listed in SEQ ID NO: 52-74. The skilled person will be able to determine the best combinations of CDRs disclosed. The VHH antibody preferably comprises a variable domain that binds von Willebrand factor (vWF). Binding is defined as a specific recognition with a higher affinity than background protein-protein binding. Preferably the affinity is in at least the micromolar range, preferably in the nanomolar range. The basic human VWF monomer is a 2050-amino acid protein. Every monomer contains a number of specific domains with a specific function including e.g. the D′D3 domain, which binds to factor VIII, the A1 domain, which inter alia binds to the platelet GPIb-receptor, the A2 domain (which must partially unfold to expose the buried cleavage site for the specific ADAMTS13 protease that inactivates VWF by making much smaller multimers), the A3 domain, which binds to collagen, the Cl domain, in which the RGD motif binds to platelet integrin allbp3 when this is activated and the “cysteine knot” domain (at the C-terminal end of the protein). Multimers of VWF can be extremely large, typically greater than 20,000 kDa and can consist of over 80 subunits of 250 kDa each.

The VHH of the invention can specifically bind to various forms, conformations, domains and epitopes of VWF, depending on the amino acid composition of the CDRs in the variable domain). For example the VHH of the invention can specifically bind to at least one of the unrolled (unfolded, activated) conformation of VWF and the globular (circulating un-activated) conformation of VWF. Preferably, the VHH of the invention binds preferentially unrolled VWF over globular VWF (i.e. has a higher affinity for unfolded VWF than for globular VWF). Unrolled VWF exposes several important binding sites, for example the main binding domain(s) for plasmin. A preferential binding to unrolled VWF helps to improve the efficiency and efficacy of directing the urokinase catalytic domain to its desired site of action. More specifically, the VHH of the invention binds to the unrolled (activated) conformation of VWF and does not bind to circulating un-activated globular conformation of VWF. The VHH of the invention may binds specifically to one or more domains of VWF, for example the D′D3 domain, and/or the C-terminal cystine knot (CTCK) domain. Preferably, the VHH of the invention binds specifically to the CTCK domain of (unrolled) VWF.

Expressed in equilibrium dissociation constant (KD), the VHH of the present invention may have an affinity to human VWF of at least or stronger than 10 nM, at least or stronger than 5 nM, at least or stronger than 1 nM, at least or stronger than 500 pM, at least or stronger than 400 pM, at least or stronger than 300 pM. Preferably, the VHH of the present invention has an affinity to unrolled human VWF of at least or stronger than 250 pM, at least or stronger than 200 pM, at least or stronger than 150 pM. More preferably, the VHH of the present invention has an affinity to unrolled human VWF of at least or stronger than 100 pM. In one preferred embodiment, the VHH of the present invention has an affinity to unrolled human VWF of at least or stronger than 90 pM, for example 70 pM.

The sequence encoding the VHH preferably is codon optimized, preferably comprising encoding amino acid changes for humanization and/or deimmunization of the VHH. The terms “humanization” and/or “deimmunization” refer to a modification wherein amino acids of an antibody derived from a non-human source (e.g., camelid, mouse, or other species) are modified to make them more similar to human antibodies. The goal of humanization is to reduce the potential immunogenicity (the likelihood of eliciting an immune response) when the antibody is used in humans for therapeutic or diagnostic purposes. When an antibody is humanized, one or more amino acid residues are replaced by one or more of the amino acid residues that predominantly occur at the corresponding position(s) in a VH domain from a conventional 4-chain antibody from a human being. The hybrid protein, as set out above can be either a fusion protein or a chemically linked protein.

The hybrid protein of the present invention preferably also comprises a linker-amino acid sequence located between the VHH on the one hand, the urokinase plasminogen activator, on the other hand. A preferred linker-amino acid sequence for linking the targeting agents and the plasminogen activator has the sequence of (Gly-Gly-Gly-Gly-Ser) n, wherein n represents an integer (e.g. 1, 2, 3, etc.). The term “fusion protein” is defined as understood by a person skilled in the art and refers to proteins that are created through the joining of two or more genes that originally coded for separate proteins. The term “chemically linked protein” is defined as understood by a person skilled in the art as a construct where two or more proteins are covalently attached to each other using chemical methods, including the use of cross-linking agents, such as bifunctional reagents, click chemistry, or enzymatic conjugation. The hybrid protein preferably comprises (preferably in a N- to C-terminal order): the antibody or antigen binding fragment, a linker sequence, a moiety capable of facilitating the lysis of microthrombi, most preferably the hybrid protein comprises in a N- to C-terminal order: a VHH antibody, a Gly/Ser-linker sequence, and a urokinase plasminogen activator. Alternatively, the hybrid protein can comprise more than one VHH, more than one linker sequence, and/or more than one urokinase plasminogen activator. Preferably the hybrid protein according to the invention comprises or consists of the amino acid sequence of SEQ ID NO: 1, SEQ ID NO: 2, or SEQ ID NO: 3. Once administered, the hybrid protein of the present invention may be present in the plasma in free form and/or in VWF-bound form. Preferably, the hybrid protein of the present invention, when in free form, may have an effective half-life in a range from 0.25 to 9 hours, preferably, preferably 0.5 to 6 hours, for example from 0.9 to 5.7 hours; and/or a terminal elimination half life in a range from 8.5 to 15.5 hours, preferably from 9.25 to 14.5 hours. Taking the VWF-bound species into account, the hybrid protein of the present invention may have an (total) effective half-life in a range from 6 to 8 hours, preferably from 6.25 to 7 hours; and/or a (total) terminal elimination half-life in a range from 14 to 21.5 hours, preferably from 14.5 to 20.5 hours. The half-lives disclosed in this paragraph refer to metabolism of the hybrid protein in healthy human adults, without them being bound to thrombi.

The invention further provides a method for modulating, preferably reducing the risk of microvascular thrombosis, wherein the method comprises the step of administering to a subject in need thereof, an effective amount of a hybrid protein as disclosed herein above. The hybrid protein is preferably administered in a dosage up to 1.5 milligram (mg), up to 1.0 mg, up to 0.9 mg, up to 0.8 mg, up to 0.7 mg, up to 0.6 mg, up to 0.5 mg per kilogram of body weight. Preferably, the hybrid protein is administered in a dosage of at least 0.05 mg, at least 0.06 mg, at least 0.08 mg, at least 0.09 mg, at least 0.1 mg, per kilogram of body weight. For an adult patient, the hybrid protein may be administered in a dosage of 0.5 to 100 mg, preferably 1.0 to 50 mg, more preferably 2.0 to 32 mg. The hybrid protein is preferably administered via intravenous (IV) infusion or intravenous bolus. For example, the hybrid protein may be administered as a controlled IV infusion over 0.5 min to 60 min, preferably over 0.5 min to 5 min, for example over 1 min. Alternatively, the hybrid protein may be administered via IV bolus. Preferably, the hybrid protein is administered via intravenous infusion or intravenous bolus in a dosage from 0.02 to 1.5, for example from 0.04 to 1.0 mg per kg of body weight. Alternatively, the hybrid protein is administered via intravenous infusion or intravenous bolus in a dosage from 0.05 to 1.5 mg per kg of body weight. Preferably, the hybrid protein is administered to an adult human patient via intravenous infusion or intravenous bolus in a dosage from 0.5 to 50 mg, more preferably from 1 to 32 mg, from 2 to 16 mg, for example 8 mg. The hybrid protein of the present invention may be administered not more than 5 times in one week, preferably not more than 4 times in one week, not more than 5 times in two weeks, most preferably not more than 4 times in two weeks.

Preferably the microvascular thrombosis is treated or the risk of its occurrence is reduced in a disease or condition selected from a group consisting of: acquired or hereditary thrombotic thrombocytopenia purpura (TTP) complement-mediated thrombotic microangiopathy, haemolytic uremic syndrome, antiphospholipid antibody syndrome, non-occlusive thrombus, the formation of an occlusive thrombus, arterial thrombus formation, acute coronary occlusion, peripheral arterial occlusive disease, restenosis and disorders arising from coronary bypass graft, coronary artery valve replacement and coronary interventions such angioplasty, stenting or atherectomy, hyperplasia after angioplasty, atherectomy or arterial stenting, occlusive syndrome in a vascular system or lack of patency of diseased arteries, transient cerebral ischemic attack, unstable or stable angina pectoris, cerebral infarction, HELLP syndrome, carotid endarterectomy, carotid artery stenosis, critical limb ischemia, cardioembolism, peripheral vascular disease, restenosis, sickle cell disease and myocardial infarct. By providing methods of administrating an effective amount of the hybrid protein, the inventors have provided means and methods for treating or reducing the risk of microvascular thrombosis.

A further method according to the invention, for producing the hybrid protein as set out above, comprises the use of producer cells, preferably eukaryotic cells. The term “producer cells” refers to cells that are capable of synthesizing and preferably secreting large quantities of a specific recombinant protein of interest. These cells are engineered to contain a gene or genes encoding the desired protein, allowing them to produce and preferably secrete the recombinant protein into the surrounding environment. The producer cells can be of various types, such as bacteria, yeast, insect cells, or mammalian cells, depending on the specific requirements of the protein and the intended application. Preferably the producer cells are yeast cells, in particular Pichia pastoris cells or Saccharomyces cerevisiae cells, wherein Pichia pastoris cells are preferred. Alternatively the producer cells can be mammalian cells, wherein Chinese hamster ovary cells are preferred. The hybrid protein, or preferably a precursor of the hybrid protein, can further comprise a secretion signal peptide, preferably an a-mating factor (preferably according to SEQ ID No. 4), or a secretion signal peptide such as Murine IgKVIII (preferably according to SEQ ID No. 5). The term “secretion signal peptide a-mating factor” describes a signal peptide that targets a peptide to the secretory pathway of a yeast cell, preferably Pichia pastoris. The term “secretion signal peptide Murine IgK VIII” is defined as a signal peptide that is commonly used in Chinese Hamster Ovary cells that guides a peptide to the secretory pathway of the cell, ensuring proper processing and secretion. The method of producing the hybrid protein is as understood by a person skilled in the art and comprises the following steps: growth phase of the producer cells; induction phase of the producer cells; harvesting; and one or more purifying steps such as: filtration, Protein A-Sepharose purification; Hydrophobic interaction chromatography (HIC); Ion exchange (IEX) chromatography.

The invention further provides an isolated nucleic acid molecule according to the invention, encoding a hybrid protein as set out above. Preferably the isolated nucleic acid molecule is part of a circular nucleic acid molecule. The term “circular nucleic acid molecule” is defined as a nucleic acid (DNA or RNA) molecule that forms a closed loop structure, where the ends of the molecule are covalently linked together. The term “circular nucleic acid molecule” includes what is known in the art as an expression vector or expression construct, which are nucleic acid molecules that are capable of effecting expression of a nucleotide sequence or gene in host cells or host organisms compatible with such expression vectors or constructs. The circular nucleic acid molecule, further preferably comprises a promoter sequence operably linked to the hybrid protein. As used herein, the term “operably linked” refers to a linkage of polynucleotide elements in a functional relationship. A nucleic acid is “operably linked” when it is placed into a functional relationship with another nucleic acid sequence. For instance, a promoter sequence is operably linked to a coding sequence if it affects the transcription of the coding sequence. The promoter sequence can be an inducible promoter sequence or a constitutive promoter sequence, preferably the inducible promoter sequence is an Alcohol oxidase I (A0X1) promoter sequence and the constitutive promoter sequence is a cytomegalovirus (CMV) promoter sequence.

The invention further provides a pharmaceutical composition comprising the hybrid protein as disclosed above and suitable excipients for administration. The pharmaceutical composition preferably comprises sodium acetate or sodium citrate, wherein sodium acetate is most preferred. The pharmaceutical composition further preferably comprises one or more pharmaceutically acceptable excipients. The term “pharmaceutically acceptable” refers to molecular entities and compositions that do not produce adverse, allergic or other unwanted reactions when administered to a subject. Whether certain adverse effects are acceptable is determined based on the severity of the disease. The pharmaceutical composition is preferably formulated as a liquid formulation, or as a lyophilized formulation to be reconstituted with water or buffer for injection.

The pharmaceutical composition of the present invention may comprise a buffered system, for example a system commonly used in pharmaceutical formulations for the purpose of, for example, maintaining the solubility, stability, bioavailability and/or bio-functionality of the active agent. Exemplary buffer system that may be suitable for use in the pharmaceutical composition of the present invention may be a phosphate buffer (PBS), an acetate (for example sodium acetate) buffer, a citrate buffer, a tris buffer. The type and composition of the buffer system is chosen with various considerations such as the ionic strength of the composition, the excipients used, the administration route and dosage requirement, the manufacturing and storage conditions, etc. Preferably, the pharmaceutical composition of the present invention comprises a buffer having a pH in the range from 5.0 to 7.5, for example a buffer having a pH of 5.5, or a buffer having a pH of 7.4.

The pharmaceutical composition of the present invention may comprise one or more excipients selected from the group consisting of solvents, tonicity adjusters, stabilizers, solubilizers, surfactants, preservatives, and/or complexing agents.

A kit of parts according to the invention comprising the hybrid protein or a pharmaceutical composition and one of more of: tranexamic acid (TXA), plasmin-inhibitor (PI), an antibody against urokinase. Preferably one or more of the following compounds is administered to the subject to accomplish the regulating effect of the hybrid protein: tranexamic acid (TXA), plasmin-inhibitor (PI), antibody against urokinase. In other words, the effect caused by administering the hybrid protein to a subject, is reduced or stopped. By providing methods of regulating the effect of the hybrid protein, the inventors have established improved means and methods for exercising control over the treatment of a patient with the hybrid protein.

### EXAMPLES

### Example 1—Preparation of Microlyse Variant 1.0 and 1.1 (ML 1.0 and ML 1.1)

The cDNA sequence for both human and mouse urokinase (PLAU) was obtained from the NCBI database (NM 002658.4 and NM 008873.3 respectively). The sequence for the signal peptide, EGL-like and Kringle domain were removed as well as the first part of the connecting peptide. To the remaining connecting peptide and SI peptidase domain (Catalytic domain) a N-terminal sequence coding for a Tobacco Etch Virus cleavage site followed by an GGGGS linker was added. In the GGGGS linked a PstI and BamHI digestion site were incorporated without disturbing the amino acid sequence. At the 5′ side an EcoRI digestion site was added and at the 3′ side and NotI digestion was added after the STOP codon of PLAU. In the case of the Microlyse variant 1.1 (ML 1.1) arginine residue at position 153 is substituted with a glutamine residue in the Urokinase sequence prior to ordering both sequences (ML 1.0 and ML 1.1) as custom gene construct from IDT (Integrated DNA Technologies, Leuven, Belgium).

Coding sequences for nanobodies (also known as VHH) kept as the original llama sequence of humanized via 4 distinct amino substitutions within the nanobody framework (A14P, E44G, K87R, P88A). Hereafter, the sequences were codon optimized via IDT for expression in a human host cells. At the N-terminal side of the VHH coding sequence, a sequence coding for a Tobacco Etch Virus cleavage site was placed and at the C-terminal side a GGGGS linker (encoding a PstI and BamHI digestion site). These DNA segments were obtained from IDT as Double stranded DNA fragments (gBLocks).

The custom gene constructs were propagated in E. coli TOP 10 and selected by ampicillin resistance. Obtained plasmid DNAs were digested by EcoRI and NotI. The resulting inserts (886) were separated on and isolated from agarose gel and ligated into a modified pcDNA6 expression vector (pSM2) (De Maat et al, 2016 J Allergy Clin Immunol November; 30; 138 (5): 1414-23)). pSM2 encodes a N-terminal murine IgK secretion signal and a double STREP isolation tag whereafter the modified UPA constructs were ligated.

The gBlocks were ligated into the p JET 1.2 cloning vector according to manufacturer instructions (Clone JET PCR Cloning Kit; Thermo Fisher). The constructs were propagated in E. coli TOP 10 and selected by ampicillin resistance. Obtained plasmid DNAs were digested by EcoRI and BamHI. The resulting insert was separated on and isolated from agarose gel and ligated into the pSM2 vector containing the miniUPA construct.

The ML1.0s ML1.1-pSM2 constructs were transfected into HEK293 FreeStyle™ cells using 239Fectin as instructed by the manufacturer (ThermoFisher) in 6-well plates. After 7 days of production samples were harvested and centrifugated at 2000×g for 10 minutes. Supernatant contain ML1.0/ML1.1 was separated from the biomass and centrifugated again to remove the final cellular debri. Microlyse titers were determined via the VWF binding ELISA as previously published (De Maat et al, 2022 Blood January; 27; 139 (4): 597-607)). Titers of Microlyse variants were equalized via the dilution with supernatant of mock-transfected HEK293 FreeStyle™ cells.

### Example 2—Susceptibility of Microlyse Variants ML 1.0 and ML 1.1 to Cleavage by Thrombin

20 pL of Micro lyse variant supernatant obtained from Example 1 was incubated with 5 pL of vehicle or Thrombin (prediluted in 0.2% w/v BSA-HBS) and incubated at 37° C. for 30 minutes. Hereafter the reaction was stopped via the addition of reducing sample buffer (25 Mm DTT). Samples were heated to 95° C. for 10 minutes and subsequently separated on a 4-12% gradient Bis-Tris gel in MES buffer. The gel is transferred onto a Immobilon-FL membrane in blotting buffer for 1 hour at 125 Volt. The membrane is blocked with 0.5× Odyssey blocking buffer where after the constructs are detected with a rabbit polyclonal anti human UPA antibody in combination with an IR800 labeled Goat-anti-Rabbit antibody. Results were analyzed via the near-infrared odyssey scanner (Licor) according to manufacturer instructions.

In FIG. 1 results are shown indicating susceptibility of Micro lyse variants ML 1.0 and ML 1.1 to cleavage by thrombin. ML 1.1 contains a glutamine at position 153, while ML 1.0 contains an arginine at position 153. For both ML 1.0 and ML 1.1 a distinct band at around 35 Kd is observed, indicating cleavage of Microlyse by thrombin.

### Example 3—PreDeFT Immunogenicity Analysis

In silicon evaluation was performed for sequences of ML 1.0 and ML 1.1 for their immunogenic potential in humans.

### Methods

The amino acid sequences of ML 1.1 and ML 1.0 were screened for the presence of Class II (HLA-DR) restricted HLA ligands or putative T cell epitopes using the EpiMatrix system. The sequence subunits were evaluated for global and regional immunogenic potentials. To determine whether any of the putative T cell epitopes identified by EpiMatrix are homologous to predicted epitopes found within the human proteome, and thereby less likely to drive anti-therapeutic immune response, all the putative epitopes identified by EpiMatrix were screened against the human proteome using the JanusMatrix algorithm. Further, to identify any predicted T cell epitopes which may have been previously studied and evaluated, all putative T cell epitopes identified by EpiMatrix were screened against a version of the Immune Epitope Database (IEDB). The information was collated to provide an EpiMatrix score as a measure of immunogenic potential.

### Results

Regional analysis revealed 12 putative T cell epitope clusters; five of these are derived from the VhH domain and seven are derived from the UPA domain. Of the 12 putative T cell epitope clusters identified, six clusters (9-31, 33-48, 57-79, 76-94 of the VhH domain; 192-207 and 249-267 of the UPA domain) are expected to be tolerated by the human immune system and, under normal circumstances, may be actively tolerogenic in at least some human subjects. The remaining six T cell epitope clusters score high on the EpiMatrix scale and low on the JanusMatrix scale and one cluster (44-58) overlaps an antibody complementarity determining region (CDR). Under normal circumstances, it is possible that these peptides could be seen as foreign by the human immune system and may help to induce anti-therapeutic immune response.

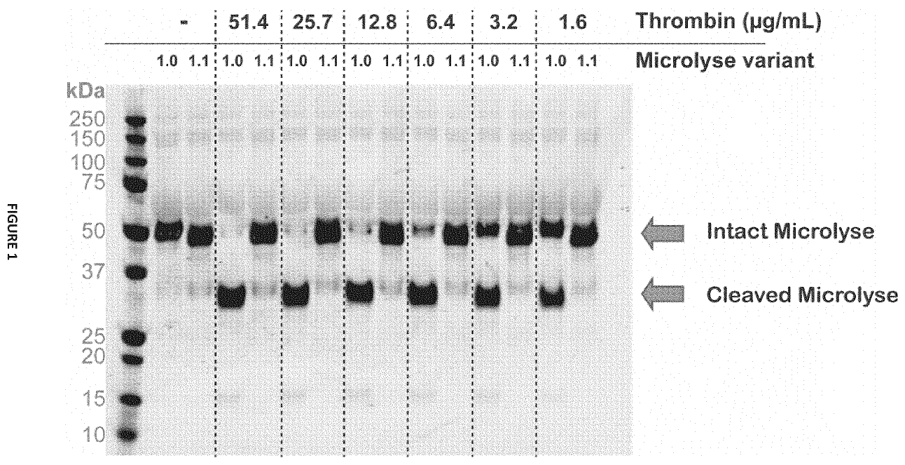

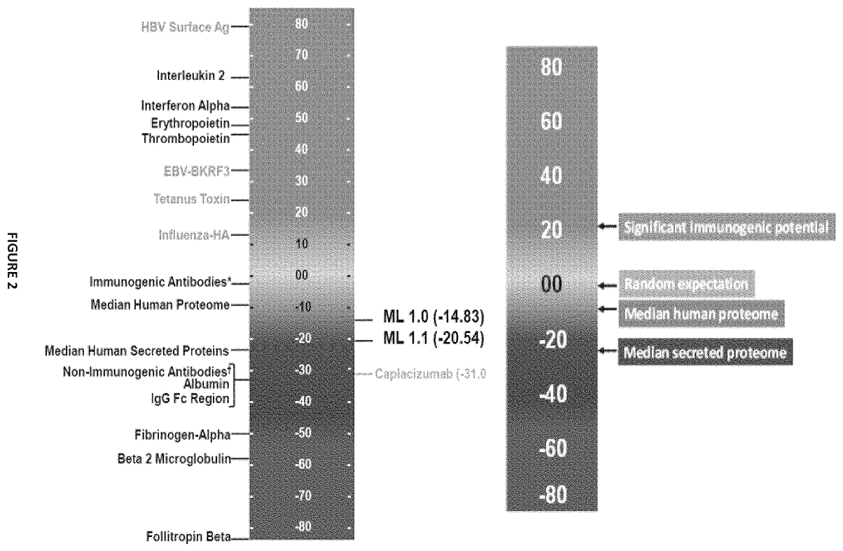

At a global level, the Tregitope-adjusted EpiMatrix Score of the ML 1.1 (−20.54) falls in the low range on the EpiMatrix Protein Immunogenicity Scale (FIG. 2), suggesting a minimal potential for driving anti-therapeutic immune response. As expected, the humanization of the VHH domain had reduced the immunological potential of ML 1.1 compared to ML-CTCK-hUPA (−14.83; FIG. 2). Investigation of ML 1.1 domains revealed that the VHH domain (−25.16) and linker (−25.58) fell in the low range, while the catalytic domain of UP A fell in the low-neutral range (−13.97).

### Conclusion

ML 1.1 falls in the low range on the EpiMatrix Protein Immunogenicity Scale, suggesting a minimal potential for driving anti-therapeutic immune response in humans.

### Example 4—Mass Spectrometry Analysis of ML 1.0 and ML 1.1

ML 1.0 used in this example was transiently expressed in HEK cells (i.e. as described in Example 1). It is further purified via the Streptavidine tag as previously published (De Maat et al, 2022 Blood January; 27; 139 (4): 597-607). The amino acid sequence of ML 1.0 with IgG secretion signal (cleaved off during secretion) and an N-terminal purification tag is described in SEQ ID NO. 102.

ML 1.1 used for this example was prepared as described in Example 1, except that it was recombinantly expressed in Pichia pastoris cells. The amino acid sequence of ML 1.1 is described in SEQ ID NO. 1.

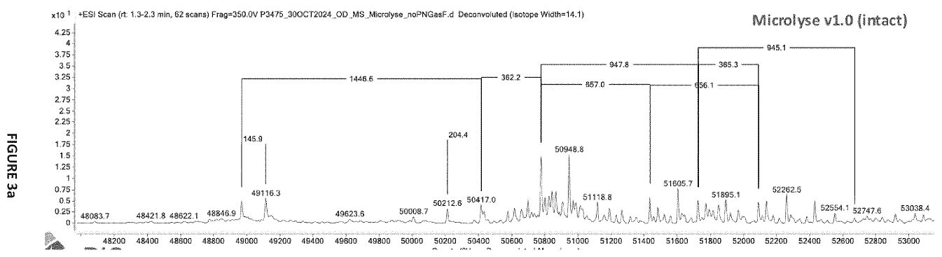

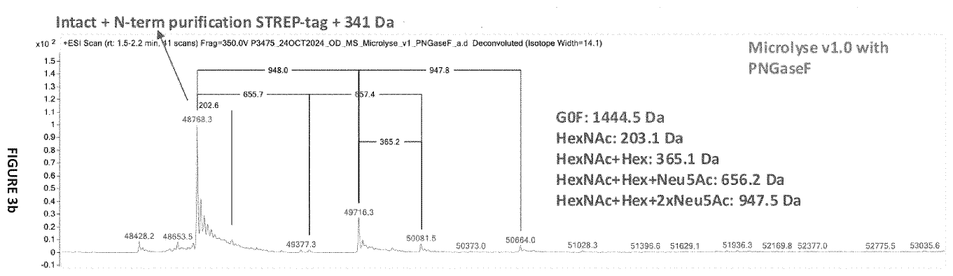

The ML 1.0 obtained was analyzed with high resolution mass spectrometry (MS) analysis. FIGS. 3a and 3b are the deconvoluted MS spectra of ML1.0 before and after treatment with PNGase F, respectively. In the PNGase treated sample the predominant mass measured is as expected that of the ML 1.0 with the N-terminal purification tag attached. The measured mass is 48768.3 Da and is 341 Da higher than expected from the amino acid sequence. This is most likely due to the presence of biotin being present in the sample. This is not unexpected since the protein has been purified via the N-terminal streptavidine tag. Hence, it is concluded that the correct sequence is found back and all N-linked glycosylations have been removed. In FIG. 3a, it is shown that the spectra of the non-deglycosylated ML 1.0 contains several mass variants (shift to higher masses) that can be attributed to the presence of both N-glycans and sialylated core 1 type O-glycans. However, the deconvolution of MS is complex due to the presence of many mass variants (combinations of N-glycans with several O-glycans). Nevertheless, it could still be concluded that ML1.0 contains N-linked glycosylations.

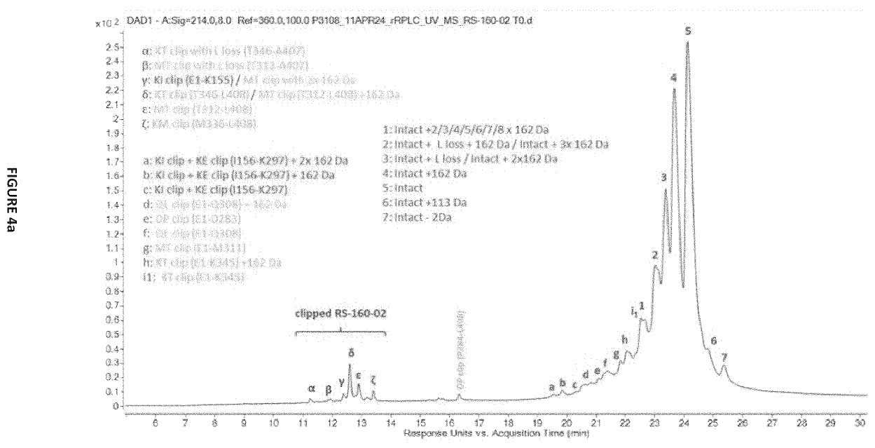

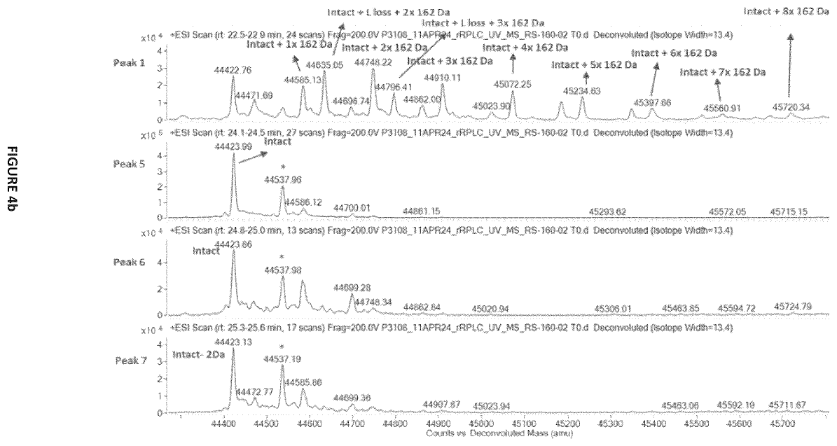

With ML 1.1, the host Pichia pastoris is capable of N-glycosilation. Since the amino acid sequence of ML1.1 is devoid of N-glycosilation sites, no N-glycosilation is expected. The ML 1.1 obtained was analyzed with Reverse Phase High-Performance Liquid Chromatography Mass Spectrometry (RP-HPLC-MS). The Result is shown in FIG. 4a. The identity of each peak as described in the figure legend was confirmed by High-Resolution Mass Spectrometry (HR-MS) as shown in FIG. 4b. Peaks labeled with (*) are most likely TFA (trifluoroacetic acid) adducts.

The analysis confirmed that ML 1.1 expressed in Pichia pastoris cells does not carry any N-glycosilations. The accurate mass measured by HR-MS corresponds to the expected molecular mass. It was also shown that about 40% of ML1.1 carry one up to three mannoses and trace amounts of up to five mannoses.

### Example 5—Repeat Dose Toxicology Study

ML 1.1 used for this example was prepared as described in Example 1, except that it was recombinantly expressed in Pichia pastoris cells and therefore does not carry any N-glycosilations. For this Example, ML1.1 was formulated in an aqueous buffer (pH 5.0) to a 2 mg/mL solution for parenteral application.

Repeat dose toxicity study was performed according to Good Laboratory Practice (GLP) principles in a rodent (rat) species. The objective of this study was to determine the toxicity of ML 1.1 following administration by the IV bolus route to the Wistar Han IGS rat for 4 weeks. In addition, the toxicokinetic characteristics of ML 1.1 were evaluated. Plasma pharmacokinetics (PK) evaluation was included in the study. AD As were analyzed in rat plasma samples. Safety pharmacology was also evaluated as part of the study.

An overview of the study performed with ML 1.1 is presented in Table 1 below.

TABLE 1
Toxicology study for ML 1.1

| | | | Frequency and | |
|---|---|---|---|---|
| | Species | | total number of | Dose levels |
| Type of Study | (Strain) | Route | administrations | (mg/kg/day) |
| Pivotal 4-week | Rat (Wistar | IV | Seven | 0, 1, 4, 16 |
| tox + 2-week | Han) | Bolus | administrations: | |
| recovery | | | Days 1, 3. 5, 8, | |
| | | | 15, 22, 29 | |

Note:
Tox = toxicology; PK = pharmacokinetic; IV = intravenous.

Male and female rats (10 animals/sex/group) received a bolus injection (flow rate: 1 mL/min; dose volume: 8 mL/kg) at dose levels of 0, 1, 4 and 16 mg/kg. Animals received ML1.1 three times the first week and weekly thereafter (a total of 7 administrations). Terminal sacrifice was performed on Day 30, one day after the last dose. Blood samples for TK evaluation were taken from a satellite group of rats (6 animals/sex/group) for up to 8 hours on Day 1 and 29.

Assessment of toxicity was based on mortality, clinical observations, body weights, body weight gains, clinical pathology parameters (hematology, coagulation, clinical chemistry), organ weights, behavioral changes and macroscopic and microscopic examinations.

Systemic exposure to ML 1.1 was confirmed on Days 1 and 29. Overall, there were no adverse pathology findings and no ML 1.1 related target organ effects were evidenced. In particular, there was no premature death, no ML 1.1-related ophthalmic findings at the end of the 4-week dosing period, and no ML 1.1-related changes in hematology, clinical chemistry and coagulation parameters. Furthermore, there was no ML 1.1-related clinical signs, no direct ML 1.1-related gross findings or organ weight changes at any dose level.

In conclusion, administration of 1, 4, and 16 mg/kg of ML 1.1 by IV (bolus) route for 4 weeks (3 times the first week and weekly thereafter, a total of 7 administrations) was well tolerated in Wistar Han IGS rats. No target organ effects were evidenced. The IV administration was well tolerated locally. Based on these results, the no observed adverse effect level (NOAEL) was considered to be 16 mg/kg. At the NOAEL, the mean Co and AUCtiast values for males were 263 μg/mL and 52.8 pg×h/mL, and for females 170 μg/mL and 35 pg×h/L, respectively, after the first dose on Day 1.

### Example 6—Tail-Clip Bleeding Experiments

Methodology ML 1.1 used for this example was prepared as described in Example 1, except that it was recombinantly expressed in Vic iapastoris cells. For this Example, ML1.1 was formulated into solution for parenteral application in an aqueous buffer (pH 5.0). The concentration of ML 1.1 in the formulation is 2 mg/mL or as otherwise specified in the Experiments below. The formulation is referred to as “TGD001” in the corresponding figures.

The tail-clip bleeding assay was conducted in adult wild-type strain mice (at least n=3 per group) at the animal institution of KU Leuven (Kortrijk, Belgium). Mice were anesthetized using an IP injection of 10 mg/kg Xylazine+100 mg/kg Ketamine. A 5 mm segment was amputated from the distal end of the tail using a scalpel. The tail was immediately immersed in 0.9% NaCl at 37° C. The time to cessation of blood loss was monitored for up to 600 seconds.

The effects of several variables were assessed in five experiments, see Table 1 for an overview of the experimental setups. In experiment 1, the tail-clip model was validated using a recombinant human tissue plasminogen activator (rh-tPA; Alteplase). Mice received an infusion (10% bolus+90% infusion over 40 minutes) of 1 or 2 mg/kg of Alteplase. The tailclip was conducted at 20 or 40 minutes after the start of treatment. In experiment 2, ML 1.1 (2.0 mg/kg) was given as a 10% bolus followed by a 40-minute infusion (remaining 90%). The tail-clip assay was performed at 20 (during the infusion) and 40 minutes (end of infusion) after treatment initiation. A bolus administration of a ML 1.1 dose range was assessed in experiment 3. Mice received a bolus injection of ML 1.1 (0.25, 1, 2 mg/kg) and the tail-clip was performed directly after treatment. Experiment 4 investigated the effect of ML 1.1's spontaneous activity on bleeding time in the tail-clip model. Mice received either a bolus administration of a ML 1.1 sample with a high level of spontaneous intrinsic activity caused by a variation in downstream processing (freeze-thaw in PBS) or standard ML 1.1 (batch) with (10% spontaneous activity (both 2 mg/kg). Experiment 5 investigated the effect of tranexamic acid (TXA), a commercially available antifibrino lytic agent, on the thrombolytic activity of ML 1.1. Mice received a bolus ML 1.1 injection (2 mg/kg) with a TXA infusion started directly after the ML 1.1 administration.

TABLE 2
The experimental setups of each experiment

| Experiment | Treatment | Dose | Tail-clip timing |
|---|---|---|---|
| 1 | Alteplase (rh-tPA) 10% bolus followed by | 1 or 2 mg/kg | 20 or 40 minutes after |
| | a 90% infusion over 40 minutes | | treatment start |
| 2 | ML1.1 10% bolus followed by a 90% | 2 mg/kg | 20 or 40 minutes after |
| | infusion over 40 minutes | | treatment start |
| 3 | ML1.1 bolus | 0.25, 1, or 2 mg/kg | Immediately after |
| | | | treatment administration |
| 4 | ML1.1 with <10% spontaneous activity or | 0.25, 1, or 2 mg/kg | Immediately after |
| | fully active ML1.1 | | treatment administration |
| 5 | ML.1.1 bolus with or without TXA infusion | ML1.1: 2 mg/kg | Immediately after |
| | (a total of 25 minutes*) | TXA: 100 mg/kg | treatment administration |

Note.
rh-tPA = recombinant human tissue plasminogen activator;
TXA = tranexamic acid:
*TXA infusion stared 15 minutes prior to the ML1.1 bolus/tail-clip. Infusion was continued during the bleeding time to a maximum of 10 minutes after which the mice were terminated)

### Result and Discussion

### Experiment 1: Alteplase Infusion

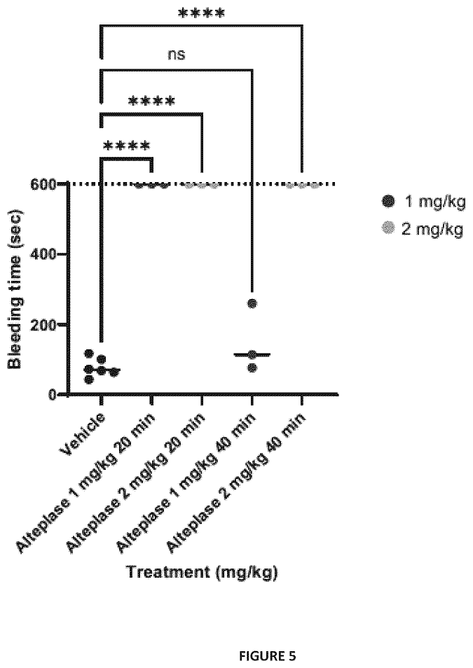

To characterize the tail-clip model, the effect of Alteplase (recombinant human tPA; rh-tPA) treatment was first tested. Alteplase has a very short half-life (6 minutes in humans) and therefore requires a 10% bolus and 90% infusion to generate sufficient exposure to initiate thrombolysis. Therefore, mice were infused with 1 or 2 mg/kg of Alteplase and compared to vehicle (0 mg/kg). For vehicle, mice showed a normal bleeding time with an average of 77.2 (standard deviation: 26.8) seconds, but when treated with Alteplase, mice showed maximum bleeding time of 600 seconds at 20 minutes post the start of infusion (i.e. mid-way through infusion). Interestingly, when the experiment was repeated but the tail-clip was initiated at the end of infusion (40 minutes) the 1 mg/kg Alteplase showed a reduced increase in bleeding time (not significantly different to the vehicle group), while the 2 mg/kg still showed a maximum bleeding time of 600 seconds (FIG. 5). These data confirmed that the tail-clip assay was sensitive to thrombolytic activity by a plasminogen activator. Recombinant human tissue plasminogen activator has previously been shown to prolong bleeding times in the tail-clip assay (Danielyan et al., 2008; Jankun et al., 2010). It should be noted that in our mouse efficacy models a 10 mg/kg dose of Alteplase is used, and has not caused spontaneous bleeding tendencies. Therefore, the tail-clip model does not necessarily predict spontaneous bleeding risk in vivo.

### Experiment 2: ML 1.1 Infusion

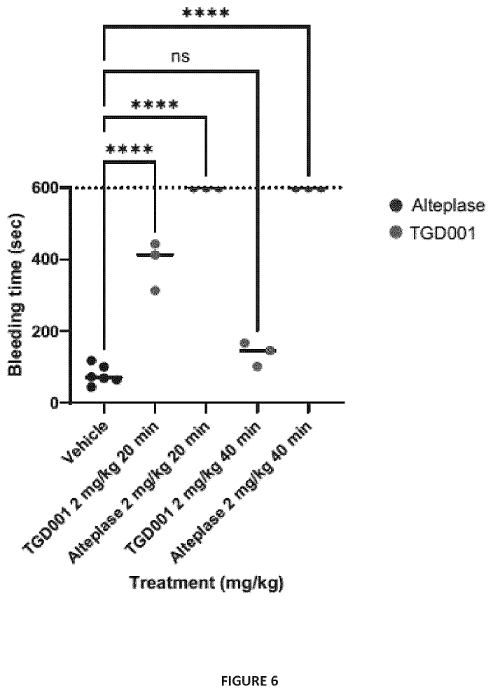

In a follow-up experiment, the effect of ML 1.1 was tested in the tail-clip model at 2 mg/kg following the same infusion strategy as Alteplase to allow for comparison. As with Alteplase, an increase in bleeding time is observed for ML 1.1 at the 20-minute time point (mid-way through infusion; PO.OOOl, FIG. 6). Interestingly, compared to tPA, ML 1.1 did not increase the bleeding time to the same extent as Alteplase (FIG. 6). Furthermore as observed with Alteplase, the bleeding time was lower when the tail-clip was performed at the end of infusion (40 minutes) and not significantly different to the vehicle group (FIG. 6). This indicates that ML 1.1's thrombolytic activity initiates rapidly upon the start of infusion and declines within 40 minutes.

### Experiment 3: Bolus ML 1.1

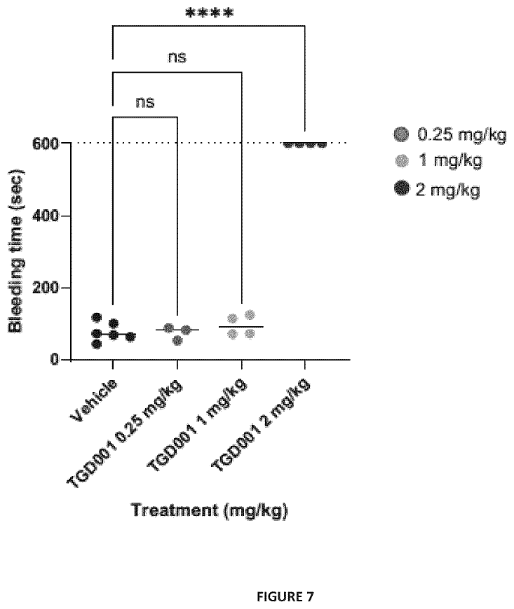

The effect of bolus administration on the tail-clip model was also tested, with the tail-clip started directly after the bolus injection.

Where mice treated up to 1 mg/kg showed no increase in bleeding time, at 2 mg/kg the bleeding time increased to the maximum of 600 seconds (FIG. 7). It is suggested that at doses where ML 1.1 demonstrates efficacy, the bleeding potential in response to large trauma is not affected.

It is believed that the extended bleeding time in the tail-clip assay does not denote spontaneous bleeding events in vivo, but rather the intended thrombolytic activity disrupting the formation of a thrombus to stop bleeding.

### Experiment 4: ML 1.1's Spontaneous Activity

ML 1.1 is produced in a zymogen form that shows very limited enzymatic activity. Only when ML 1.1 binds to its target (VWF) alongside plasminogen, the limited activity is sufficient to induce the first plasmin formation. Plasmin can then activate ML 1.1 allowing for maximal, but local plasmin formation. As such, the high zymogen concentration of ML 1.1 is essential to limit progression to systemic activation, as is often seen by completely activated urokinase.

To investigate the difference in zymogen or activated ML 1.1, a batch of ML 1.1 (formulated in PBS) was frozen and thawed to initiate auto-activation and compared to a standard batch of ML 1.1, with high levels of zymogen ML 1.1, in the tail-clip model.

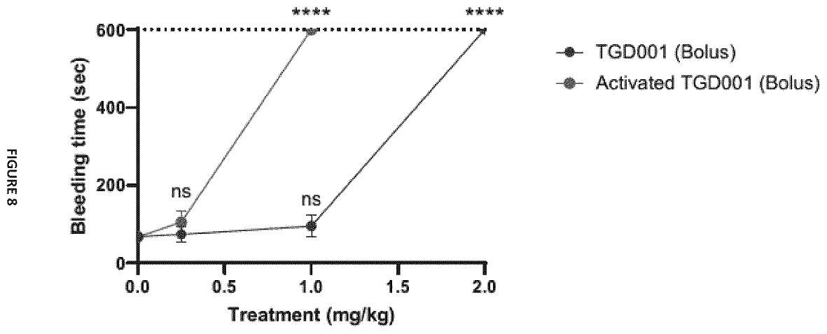

A bolus injection of ML 1.1 with high levels of spontaneous or intrinsic activity caused prolonged bleeding times at 1 mg/kg (FIG. 8). The standard ML 1.1, which has less than 10% spontaneous activity (zymogen state), prolonged bleeding times at 2 mg/kg but not 1 mg/kg. These data suggest that activated ML 1.1 has higher thrombolytic activity compared to its zymogen form. As such it might more easily lead to potential bleeding problems.

Experiment 5: ML 1.1 and TXA While thrombolytics such as rh-tPA have shown value in the clinic, their potential to induce bleeding is a concern for clinicians. While no bleeding tendencies were observed in any of our preclinical models including the GLP-toxicology studies with doses up to 16 mg/kg of ML 1.1 in the rats, an intervention strategy could be of value.

Patients with hyperfibrinolysis show bleeding tendencies. Here the plasmin formed outcompetes its natural inhibitors allowing for continuous and systemic plasmin activity. To limit the plasminogen/plasmin activity in these patients, they are treated with lysine analogues such as tranexamic acid (TXA). Both plasminogen and plasmin bind to their targets such as fibrin and VWF in a lysine-dependent manner. Lysine analogues compete with binding to plasmin (ogen)'s natural targets, thereby preventing plasminogen from becoming activated or plasmin from interacting with its targets. As ML 1.1 functions via the formation of plasmin, TXA might also be considered as a potential reversal agent.

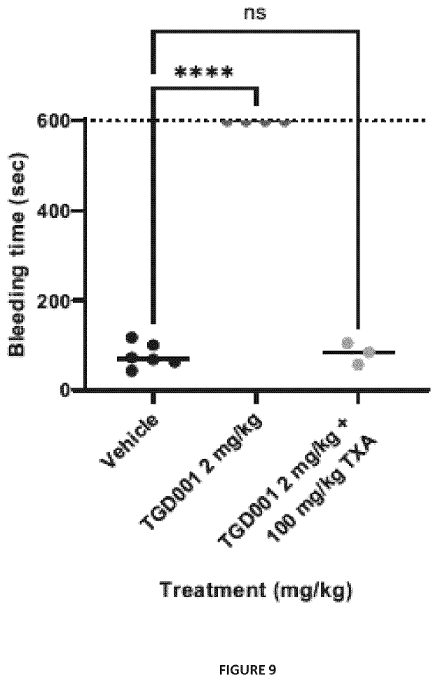

To test the effect of TXA, mice were treated with a TXA infusion directly after a 2 mg/kg ML 1.1 bolus administration. In the absence of TXA, the mice showed bleeding times over 600 seconds, while in the presence of TXA, the bleeding time returned to normal (FIG. 9). This indicates that TXA is effective at limiting ML 1.1's activity by intervening in the formation of plasmin.

### Conclusion

Thrombolytic activity, sufficient to disrupt thrombus formation, was evident in the tail-clip assay after treatment with Alteplase (infusion) and ML 1.1 (bolus and infusion) in a concentration-dependent manner. For ML 1.1, thrombolytic activity affected bleeding times within minutes after a bolus administration and declined within 40 minutes following an infusion. Higher levels of spontaneous intrinsic activity increased the thrombolytic activity of ML 1.1, while TXA infusion silenced ML 1.1's thrombolytic effect.

Interestingly, the infusion data shows lower bleeding times for 2 mg/kg ML 1.1 compared to the 1 mg/kg Alteplase. It is believed that the extended bleeding times noted in the tail-clip assay do not equate to spontaneous bleeding events in vivo, but rather are a measure of the intended thrombolytic activity disrupting the formation of the thrombus needed to stop the bleeding.

1. A polypeptide comprising a urokinase catalytic domain, wherein the arginine residue corresponding to position 153 in SEQ ID No. 1 is substituted with a glutamine residue.

2. The polypeptide according to claim 1, wherein the urokinase catalytic domain further comprises a substitution of the asparagine residue corresponding to position 299 in SEQ ID No. 1.

3. The polypeptide according to claim 2, wherein the asparagine residue corresponding to position 299 in SEQ ID No. 1 is substituted with a glutamine residue.

4. The polypeptide according to claim 1, wherein the urokinase catalytic domain further comprises a lysine residue or a histidine residue at position 297 in SEQ ID No. 1.

5-6. (canceled)

7. The urokinase catalytic domain of claim 1, that is capable of facilitating the dissolvement of microthrombi.

8. A hybrid protein comprising a polypeptide comprising a urokinase catalytic domain, wherein an arginine residue corresponding to position 153 in SEQ ID No. 1 is substituted with a glutamine residue;
wherein position 299 in SEQ ID No. 1 is an asparagine residue or a histidine residue; and
wherein the hybrid protein comprises an VHH .

9. (canceled)

10. The hybrid protein according to claim 8, wherein the VHH comprises complementarity determining regions (CDRs) CDR1, CDR2, and CDR3, wherein CDR3 comprises at least one of the amino acid sequences SEQ ID NO: 6-28;
CDR 1 comprises at least one of the amino acid sequences SEQ ID NO: 29-51; and/or
CDR2 comprises at least one of the amino acid sequences SEQ ID NO: 52-74.

11-13. (canceled)

14. The hybrid protein according to claim 8, wherein the variable domain comprises or consists of a sequence selected from SEQ ID NO: 75-101.

15. A hybrid protein according to claim 8, wherein the variable domain has binding specificity for von Willebrand factor.

16. The hybrid protein according to claim 8, wherein at least part of the coding sequence is codon optimized.

17-18. (canceled)

19. A hybrid protein according to claim 8, which is a chemically linked protein or is a fusion protein.

20. (canceled)

21. A hybrid protein according to claim 8, further comprising a linker sequence linking the polypeptide comprising the urokinase catalytic domain to the VHH.

22-23. (canceled)

24. A hybrid protein according to claim 8, which comprises the amino acid sequence of SEQ ID NO: 1, SEQ ID NO: 2, or SEQ ID NO: 3.

25. A hybrid protein according to claim 8, for use in the prevention or treatment of microvascular thrombosis.

26. A hybrid protein for use according to claim 25, which has binding specificity for von Willebrand factor.

27. A method of treatment for or reducing the risk of micro vascular thrombosis, wherein the method comprises the step of administering to a subject in need thereof, an effective amount of hybrid protein according to claim 8.

28. (canceled)

29. An isolated nucleic acid molecule encoding a hybrid protein as defined in claim 8.

30-32. (canceled)

33. A pharmaceutical composition comprising a hybrid protein according to claim 8 further comprising one or more pharmaceutically acceptable excipients.

34. The pharmaceutical composition according to claim 33, wherein the pharmaceutical composition is formulated as a liquid formulation, or as a lyophilized formulation.

35. A kit er kit of parts comprising:
the hybrid protein as defined in claim 8 and one of more of a) tranexamic acid (TXA); b) plasmin-inhibitor (PI); c) an antibody against urokinase; or
a pharmaceutical composition comprising a hybrid protein according to claim 8, one or more pharmaceutically acceptable excipients, and one of more of a) tranexamic acid (TXA); b) plasmin-inhibitor (PI); c) an antibody against urokinase.

36. (canceled)
